(19) United States
(12) Patent Application Publication
(10) Pub. No.: US 2026/0028678 A1
(43) Pub. Date: Jan. 29, 2026

(54) ASSESSMENT OF BIOLOGICAL SAMPLES FOR NUCLEIC ACID ANALYSIS

(71) Applicant: Belgian Volition SRL, Isnes (BE)

(72) Inventor: Theresa K. Kelly, Isnes (BE)

(21) Appl. No.: 19/140,988

(22) PCT Filed: Dec. 19, 2023
(86) PCT No.: PCT/EP2023/086576

§ 371 Date: Jun. 18, 2025

Related U.S. Application Data
(60) Provisional application No. 63/476,134, filed on Dec. 19, 2022.

(51) Int. Cl. C12Q 1/6886; G01N 33/574

(52) U.S. Cl. CPC C12Q 1/6886; G01N 33/57488

(57) ABSTRACT

The invention relates to the measurement of cell free nucleosomes for the selection of biological samples for DNA sequencing. The invention also relates to using the measurement of cell free nucleosomes to determine the volume of body fluid sample required to obtain a required level of DNA for DNA sequencing.

### FIELD OF THE INVENTION

The present invention relates to the measurement of cell free nucleosomes for the selection of biological samples for DNA sequencing. The invention also relates to using the measurement of cell free nucleosomes to determine the volume of body fluid sample required to obtain a required level of DNA for DNA sequencing.

### BACKGROUND OF THE INVENTION

DNA sequencing of patient samples has revolutionized the scope for personalized medicine in the care of many conditions. Patient biopsy material as well as a variety of body fluids including blood, plasma or serum may be used as a substrate for genetic DNA sequencing or epigenetic DNA sequencing (e.g. for sequences containing 5-methylcytosine).

Sequencing of DNA extracted from tissue samples is routine practice in the care of patients with a variety of conditions. The sequencing of cell free DNA (cfDNA), particularly circulating cfDNA extracted from plasma samples, is also useful for example for the emergence of resistance mutations to cancer treatment and the detection of cancer associated DNA mutations as a biomarker of cancer.

The analysis of cfDNA is particularly useful where the cell or tissue of origin can be established, for example to identify the tissue affected by a cancer. Methods of identifying the tissue of origin of cfDNA are described in the art including “fragmentomics” and “DNA-methylation” analysis.

DNA abnormalities are characteristic of all cancer diseases. The DNA of cancer cells differs from that of healthy cells in many ways including, but not limited to, point mutations, translocations, gene copy number, micro-satellite abnormalities, DNA strand integrity, nucleotide modifications (for example loss of methylation of cytosine at position 5), aneuploidy, nucleosome fragmentation patterns (fragmentomics) and others.

Tumour-associated-alterations in DNA structure or sequence are investigated routinely in cancer cells or tissue removed at biopsy or surgery for clinical diagnostic, prognostic and treatment selection purposes. Tumour genetic and epigenetic characteristics vary between different tumour types and between different patients with the same tumour disease. Moreover, these characteristics vary over time within the same cancer of the same patient with the progression of the disease and in the development of acquired resistance to drug or other therapies. Thus, investigation of tumour DNA in cells removed at surgery or biopsy may help the clinician to assess minimal residual disease, predict patient prognosis, select appropriate treatments for the patient, monitor disease progression and detect any relapse or acquired treatment resistance at an early stage (possibly many months earlier than radiological detection) and allow potentially successful changes in treatment courses.

However, tissue DNA tests have limitations as invasive biopsy procedures and cannot be performed repeatedly on patients. For some patients, biopsy may not be used at all. Biopsy is expensive to perform, uncomfortable for the patient, poses patient risk, and may lead to surgical complications. Moreover, a tumour in a patient may consist of multiple tumoural clones located within different areas of the same tumour or within different metastases (in metastatic cancer) not all of which may be sampled by biopsy. A tissue biopsy DNA investigation therefore provides a snap-shot of the tumour, both in time and in space, amongst different tumour clones located within different areas of a tumour at one particular moment in time.

As DNA abnormalities are characteristic of all cancer diseases and circulating tumour DNA (ctDNA) has been observed for cancer diseases, ctDNA tests have potential applicability in cancer diseases. Cancers investigated include, without limitation, cancer of the bladder, breast, colorectal, melanoma, ovary, prostate, lung, liver, endometrial, ovarian, lymphoma, oral, leukaemias, head and neck, and osteosarcoma (Crowley et al, 2013; Zhou et al, 2012; Jung et al, 2010).

Early approaches in ctDNA tests involved the detection of a cancer associated gene sequence mutation in ctDNA. Blood tests involving the detection of a single gene mutation in ctDNA from majority of cancer types generally has low clinical sensitivity. There are two reasons for this. Firstly, although all cancers have mutations, the frequency of any particular mutation in a particular cancer disease can be low. For example, although KRAS and p53 mutations are regarded as two of the more frequent cancer mutations and have been studied in a wide range of cancers including bladder, breast, colon, lung, liver, pancreas, endometrial and ovarian cancers, they were detected in 23%-64% and 17%-54% of cancer tissue samples respectively (Jung et al, 2010). Secondly, even if the cancer tissue of a patient does contain the mutation, the level or concentration of mutated ctDNA present in the blood of the patient may be low and difficult to detect. For example, KRAS and p53 mutations could be detected in the ctDNA of 0%-75% of KRAS and p53 tissue positive patients. The sum of these two effects meant that KRAS or p53 mutations were detected in the blood of less than 40% of cancer patients (Jung et al, 2010). Thus analysis of cfDNA for a single point mutation has not been used clinically for the routine detection of cancer.

Although mutations of any particular gene such as KRAS or p53 may be present in only a minority of cancers, all cancers contain mutations. Therefore analysis of a large panel of multiple mutations should in principle facilitate the detection of most or even all tumours. One way to increase the clinical sensitivity of such tests is therefore to test for a wide range of mutations in many genes. Newman et al. have taken this approach for non-small cell lung cancer (NSCLC) and investigated 521 exons and 13 intron sequences from 139 recurrently mutated genes. The mutations studied encompassed multiple classes of cancer associated genetic alterations, including single nucleotide variation (SNV) and fusion genes. In this way the authors reported the detection of more than 95% of stage II-IV tumours and 50% of stage I tumours with 96% specificity in ctDNA blood tests (Newman et al, 2014).

Early epigenetic approaches for the detection of cancer involved analysis of cfDNA for (single) gene epigenetic alterations to particular DNA sequences. This approach can be applied to any DNA or nucleotide modification. A prime example of this approach is the detection of genes which are differentially methylated at cytosine residues in certain cancers. A large number of genes have been investigated for this purpose in a variety of cancers. A few of these are SEPTIN-9, APC, DAPK, GSTP1, MGMT, p16, RASSF1A, T1G1, BRCA1, ERα, PRB, TMS1, MLH1, HLTF, CDKN2A, SOCS1, SOCS2, PAX5, PGR, PTGS2 and RARβ2 investigated in bladder, breast, colorectal, melanoma, ovarian and prostate cancers. Typically, bisulfite conversion sequencing methods are used in which DNA is extracted from plasma and then treated with sodium bisulfite which converts unmodified cytosine residues to uracil. Sequencing, polymerase chain reaction (PCR) or other methods can then be applied to determine whether a particular methylated gene sequence is present. An illustrative example of this approach is the detection of methylated SEPTIN-9 in ctDNA for the detection of Colorectal Cancer (CRC) which was reported to detect 48% of CRC cases with a clinical specificity of 91% (Church et al, 2014).

Some of the aforementioned ctDNA analysis methods have proven useful in the management of late stage cancer for the selection of treatment methods. However, they have hitherto proved ineffective for the identification of patients with early stage cancer (Church et al, 2014). More recently, multi-focal DNA methylation patterns (rather than single gene methylation analysis) have been analysed and this has proved more promising in this respect. Moreover, the cell(s) or tissue(s) of origin of the cfDNA may be determined by comparison of the methylated cfDNA sequence patterns obtained with known patterns for various cells or tissues as described above.

Similarly, fragmentomics is reported to be useful for the detection of early stage cancer indicating that nucleosome and other protein occupancy analysis of cfDNA patterns may be used both to detect cancer and potentially to identify the organ site of the cancer (Snyder et al, 2016).

Clearly, all DNA sequencing methods require a sample containing DNA to be sequenced and there is a minimum amount of DNA required to obtain a reliable result. A major problem associated with ctDNA analysis methods is that the mutant allele fraction (MAF—the proportion of alleles at a specific genomic location which are mutant) of cfDNA in cancer patients is low. The ctDNA analytic target of the methods is diluted in a larger quantity of circulating cfDNA of hematopoietic and other origins. This means that the proportion of cfDNA constituted by tumour derived ctDNA is low which places limitations on all ctDNA analysis methods. The MAF is particularly low in early stage cancer samples and this underlies much of the difficulty in the field of cancer detection by liquid biopsy.

DNA extraction and sequencing is often performed on samples containing inadequate concentrations or absolute amounts of DNA providing poor and unreliable results. The present invention provides a rapid low cost, i.e. an effective and efficient, method for assessment of the DNA levels present in a sample prior to DNA extraction and/or sequencing. It also provides a method of assessment of the volume of sample required to be obtained from a subject to facilitate a reliable DNA sequencing result.

### SUMMARY OF THE INVENTION

According to a first aspect of the invention, there is provided a method for the analysis of DNA in a body fluid sample comprising:
(a) measuring the level of nucleosomes in a body fluid sample;
(b) determining if the level of nucleosomes measured in step (a) meets a threshold level of nucleosomes;
(c) optionally extracting the DNA from the sample if the level of nucleosomes measured in step (b) meets the threshold level of nucleosomes; and
(d) sequencing the DNA present in the sample if the level of nucleosomes measured in step (b) meets the threshold level.

According to a further aspect of the invention, there is provided a method for the analysis of DNA in a body fluid sample comprising:
(a) measuring the level of nucleosomes in sample obtained from a body fluid;
(b) determining the volume of body fluid sample required to obtain a required level of DNA for DNA sequencing using the level of nucleosomes measured in step (a);
(c) obtaining a body fluid sample of at least the volume determined in step (c);
(d) optionally extracting DNA from the sample; and
(e) sequencing the DNA present in the sample.

### BRIEF DESCRIPTION OF THE FIGURES

FIG. 1. Genetic copy number analysis for cfDNA samples obtained from subjects diagnosed with (A) Non-Hodgkin Lymphoma (NHL) or (B) prostate cancer. The results are plotted as a log base 2 ratio where a value of zero denotes a normal diploid copy number. A value above zero denotes amplification and a value below zero denotes a deletion. Results for the NHL sample show both amplifications and deletions. Results for the prostate cancer sample show no copy number alterations.

FIG. 2. Genetic copy number analysis for a cfDNA sample obtained from a subject diagnosed with sepsis. Results for the sepsis sample show no copy number alterations.

FIG. 3. H3.1-nucleosome level results for control subjects not diagnosed with a disease.

FIG. 4. Graphs showing the impact of nucleosome input level on sequencing quality. (A) 316 human plasma cell-free chromatin samples sequenced using paired-end next generation whole-genome sequencing, showing nucleosome level (ng/ml, in log scale) vs. the percentage of reads mapping. Vertical lines indicate nucleosome threshold values of 30 and 69. (B) 34 canine plasma cell-free chromatin samples sequenced using nanopore whole-genome sequencing, showing nucleosome level (ng/ml, in log scale) vs. the number of reads sequenced, normalized to the mean read count of samples on the same sequencing pool. Vertical lines indicate nucleosome threshold values of 30 and 69.

### DETAILED DESCRIPTION OF THE INVENTION

Current methods of DNA sequencing typically involve extraction of DNA from a sample followed by sequencing. One difficulty with this process is that the sample may contain insufficient DNA for sequencing. Furthermore, by the time this information is available the sample may have been utilised or exhausted and further sample may not be available. In order to overcome these difficulties, the present invention facilitates efficient and effective determination of the DNA present in a sample for sequencing prior to DNA extraction. If the sample is inadequate, the information may be used to obtain a larger volume of sample from the subject before DNA extraction or sequencing is initiated.

The majority of cfDNA present in a body fluid sample occurs as nucleoprotein complexes in the form of mononucleosomes, oligonucleosomes or poly nucleosomes (Sanchez et al, 2021). The present invention employs the inventive concept that measurement of nucleosomes in a sample will give a result that approximates to the level of DNA present.

In general terms, the present invention provides a nucleosome assay for selecting a fluid biological sample for DNA sequencing.

Thus according to one aspect of the present invention there is provided a method for the analysis of DNA in a body fluid sample comprising:
(a) measuring the level of nucleosomes in a body fluid sample;
(b) determining if the level of nucleosomes measured in step (a) meets a threshold level of nucleosomes;
(c) optionally extracting the DNA from the sample if the level of nucleosomes measured in step (b) meets the threshold level of nucleosomes; and
(d) sequencing the DNA present in the sample if the level of nucleosomes measured in step (b) meets the threshold level.

According to another aspect of the present invention there is provided a method for the analysis of DNA in a body fluid sample comprising:
(a) measuring the level of nucleosomes in a body fluid sample;
(b) determining if the level of nucleosomes measured in step (a) meets a threshold level of nucleosomes;
(c) extracting the DNA from the sample if the level of nucleosomes measured in step (b) meets the threshold level of nucleosomes; and
(d) sequencing the DNA present in the sample if the level of nucleosomes measured in step (b) meets the threshold level.

According to another aspect of the present invention there is provided a method for the analysis of DNA in a body fluid sample comprising:
(a) measuring the level of nucleosomes in sample obtained from a body fluid;
(b) determining the volume of body fluid sample required to obtain a required level of DNA for DNA sequencing using the level of nucleosomes measured in step (a);
(c) obtaining a body fluid sample of at least the volume determined in step (c);
(d) optionally extracting DNA from the sample; and
(e) sequencing the DNA present in the sample.

According to another aspect of the present invention there is provided a method for the analysis of DNA in a body fluid sample comprising:
(a) measuring the level of nucleosomes in sample obtained from a body fluid;
(b) determining the volume of body fluid sample required to obtain a required level of DNA for DNA sequencing using the level of nucleosomes measured in step (a);
(c) obtaining a body fluid sample of at least the volume determined in step (c);
(d) extracting DNA from the sample; and
(e) sequencing the DNA present in the sample.

The present invention may also be described with reference to the following general protocols.

The invention provides the following method for the analysis of DNA in a body fluid sample obtained from a subject which comprises the steps of:
(a) measuring the level of nucleosomes in the sample;
(b) using the level of the nucleosomes measured in step (a) to determine if the nucleosome level is sufficient for reliable DNA sequence analysis; and
(c) sequencing the DNA present in the sample if the amount of nucleosomes measured in step (b) is sufficient.

In one embodiment, the method comprises extracting DNA from the sample prior to sequencing, if the amount of nucleosomes is assessed to be sufficient.

Nucleosome measurements can be performed rapidly and economically. Therefore the invention provides the following method for the analysis of DNA in a body fluid sample obtained from a subject which comprises the steps of:
(a) measuring the level of nucleosomes in the sample;
(b) using the level of nucleosomes measured in step (a) to determine the volume of sample required to obtain sufficient DNA for DNA sequence analysis;
(c) obtaining the volume of sample determined in step (b);
(d) optionally extracting DNA from the sample; and
(e) sequencing the DNA present in the sample.

### Measuring and Determining Levels of Nucleosomes

The present invention employs methods for measuring the level of nucleosomes in a sample and uses this measured level to determine inter alia whether a threshold level of nucleosomes is present in the sample. References to “nucleosome” may also encompass “cell free nucleosome” when detected in body fluid samples. It will be appreciated that the term “cell free nucleosome” used throughout this document is intended to include any cell free chromatin fragment that includes one or more nucleosomes, or partial nucleosome (e.g. where one or more histone protein dimers are lost from the nucleosome structure).

Cellular DNA exists as a protein-nucleic acid complex called chromatin. The nucleosome is the basic unit of chromatin structure and consists of DNA wound around a protein complex. The DNA is wound around consecutive nucleosomes in a structure often said to resemble “beads on a string” and this forms the basic structure of open or euchromatin. In compacted or heterochromatin this string is coiled and super coiled in a closed and complex structure.

Each nucleosome in chromatin consists of a protein complex of eight highly conserved core histones (comprising of a pair of each of the histones H2A, H2B, H3, and H4). Around this complex are wrapped approximately 145 base pairs (bp) of DNA. Another histone, H1, which may be located on the nucleosome outside of the core histones, binds a further 20 bp of DNA to produce nucleosomes (or chromatosomes) containing approximately 165 bp of DNA. Histone H1 is said to act as a linker histone and the additional DNA is often referred to as “linker DNA”, i.e. the DNA connecting one nucleosome to another in chromosomes. The linker DNA separating two nucleosomes in a chromosome is sometimes longer than 20 bp and may be up to 80 bp in length.

Normal cell turnover in adult humans involves the continuous creation of cells (by cell division) and death of cells in significant numbers daily. During the process of apoptosis chromatin is broken down into mononucleosomes and oligonucleosomes some of which may be found in circulation. In addition, extracellular traps (ETs), particularly neutrophil extracellular traps (NETs), and their metabolites can comprise a major source of cfDNA in body fluids. ETs and NETs comprised of decondensed nuclear and/or mitochondrial DNA decorated with granular proteins. They may be released into the extracellular space and into body fluids by neutrophils and other cells through the ejection of cellular chromatin in a process known as NETosis. NETosis is a part of the innate immune system and occurs in response to pathogens or other tissue insults.

Under normal conditions the levels of circulating nucleosomes found in healthy subjects is reported to be low. Elevated levels are found in subjects with a variety of conditions including many cancers, auto-immune diseases, inflammatory conditions, stroke and myocardial infarction (Holdenrieder & Stieber (2009)).

The cell free nucleosome may be mononucleosomes, oligonucleosomes, a constituent part of a larger chromatin fragment or a constituent part of a NET or a mixture thereof.

Methods and uses of the invention may measure the level of (cell free) nucleosomes per se. References to “nucleosomes per se” refers to the total nucleosome level or concentration present in the sample, regardless of any epigenetic features the nucleosomes may or may not include. Detection of the total nucleosome level typically involves detecting a histone protein common to all nucleosomes, such as histone H4. Therefore, nucleosomes per se may be measured by detecting a core histone protein, such as histone H4. As described herein, histone proteins form structural units known as nucleosomes which are used to package DNA in eukaryotic cells.

It will be understood that the cell free nucleosome may be detected by binding to a component thereof. The term “component thereof” as used herein refers to a part of the nucleosome, i.e. the whole nucleosome does not need to be detected. The component of the cell free nucleosomes may be selected from the group consisting of: a histone protein (i.e. histone H1, H2A, H2B, H3 or H4), a histone post-translational modification, such as citrullination, a histone variant or isoform, a protein bound to the nucleosome (i.e. a nucleosome-protein adduct), a DNA fragment associated with the nucleosome and/or a modified nucleotide associated with the nucleosome. For example, the component thereof may be histone (isoform) H3.1 or histone H1 or DNA.

Mononucleosomes and oligonucleosomes can be detected by Enzyme-Linked ImmunoSorbant Assay (ELISA) and several methods have been reported (e.g. Salgame et al. (1997); van Nieuwenhuijze et al. (2003); Holdenrieder et al. (2001)). These assays typically employ an anti-histone antibody (for example anti-H2B, anti-H3 or anti-H1, H2A, H2B, H3 and H4) as capture antibody and an anti-DNA or anti-H2A-H2B-DNA complex antibody as detection antibody.

Circulating nucleosomes are not a homogeneous group of protein-nucleic acid complexes. Rather, they are a heterogeneous group of chromatin fragments originating from the digestion of chromatin on cell death and include an immense variety of epigenetic structures including particular histone isoforms (or variants), post-translational histone modifications, nucleotides or modified nucleotides, and protein adducts. It will be clear to those skilled in the art that an elevation in nucleosome levels will be associated with elevations in some circulating nucleosome subsets containing particular epigenetic signals including nucleosomes comprising particular histone isoforms (or variants), comprising particular post-translational histone modifications, comprising particular nucleotides or modified nucleotides and comprising particular protein adducts. Assays for these types of chromatin fragments are known in the art (for example, see WO 2005/019826).

In more detail, methods for the measurement of total cell free nucleosomes and of cell free nucleosomes including those comprising epigenetic signals in blood and other body fluids for the diagnosis of disease are known in the art and have been described in e.g. WO2013/030577, WO2013/030578, WO2013/030579 and WO2013/084002. These methods may be used to measure the levels of nucleosomes according to the present invention.

Manual ELISA (enzyme linked immunosorbent assays) methods for the measurement of nucleosomes are among the assays described in these publications. These methods are sensitive (they can detect nucleosome levels corresponding to 2 or 3 ng/ml DNA), reliable and require a few hours to complete. Automated methods are also available that can have increased sensitivity and require less than 1 hour to complete. Immunoassays as point of care assays have also been developed. Other immunoassay methods such as homogeneous immunoassay (HIA), lateral flow immunoassay and microfluidic immunoassay can be performed at the point of care and require less than 10 minutes to complete.

The threshold level used in the methods of the invention may be the level of cell free nucleosomes per se and/or an epigenetic feature of a cell free nucleosome. It will be understood that the terms “epigenetic signal structure” and “epigenetic feature” are used interchangeably herein. They refer to particular features of the nucleosome that may be detected. In one embodiment, the epigenetic feature of the nucleosome is selected from the group consisting of a post-translational histone modification, a histone isoform, a modified nucleotide and/or proteins bound to a nucleosome in a nucleosome-protein adduct. Similarly, the threshold level used in the methods of the invention may be the level of NETs or ETs per se and/or an epigenetic feature of the NETs or ETs present in the sample.

In one embodiment, the epigenetic feature of the nucleosome comprises one or more histone variants or isoforms. The epigenetic feature of the cell free nucleosome may be a histone isoform, such as a histone isoform of a core nucleosome, in particular a histone H3 isoform. The term “histone variant” and “histone isoform” may be used interchangeably herein. The structure of the nucleosome can also vary by the inclusion of alternative histone isoforms or variants which are different gene or splice products and have different amino acid sequences. Many histone isoforms are known in the art. Histone variants can be classed into a number of families which are subdivided into individual types. The nucleotide sequences of a large number of histone variants are known and publicly available for example in the National Human Genome Research Institute NHGRI Histone Database (Mariño-Ramirez et al. The Histone Database: an integrated resource for histones and histone fold-containing proteins. Database Vol. 2011. and http://genome.nhgri.nih.gov/histones/complete.shtml), the GenBank (NIH genetic sequence) Database, the EMBL Nucleotide Sequence Database and the DNA Data Bank of Japan (DDBJ). For example, variants of histone H2 include H2A1, H2A2, mH2A1, mH2A2, H2AX and H2AZ. In another example, histone isoforms of H3 include H3.1, H3.2, H3.3 and H3t. In one embodiment, the histone isoform is H3.1. In an alternative embodiment, the histone isoform is H3.3.

Therefore, in one embodiment, the nucleosomes measured are H3.1-containing nucleosomes or H3.3-containing nucleosomes, in particular H3.1-containing nucleosomes.

The structure of nucleosomes can vary by post translational modification (PTM) of histone proteins. PTM of histone proteins typically occurs on the tails of the core histones and common modifications include acetylation, methylation or ubiquitination of lysine residues as well as methylation or citrullination of arginine residues and phosphorylation of serine residues and many others. Many histone modifications are known in the art and the number is increasing as new modifications are identified (Zhao and Garcia (2015) Cold Spring Harb Perspect Biol, 7: a025064). Therefore, in one embodiment, the epigenetic feature of the cell free nucleosome may be a histone post translational modification (PTM). The histone PTM may be a histone PTM of a core nucleosome, e.g. H3, H2A, H2B or H4, in particular H3, H2A or H2B. In particular, the histone PTM is a histone H3 PTM. Examples of such PTMs are described in WO 2005/019826 (which is herein incorporated by reference).

For example, the post translational modification may include acetylation, methylation, which may be mono-, di- or tri-methylation, phosphorylation, ribosylation, citrullination, ubiquitination, hydroxylation, glycosylation, nitrosylation, glutamination and/or isomerisation (see Ausio (2001) Biochem Cell Bio 79: 693). In one embodiment, the histone PTM is selected from citrullination or ribosylation.

In one embodiment, the histone post translational modification is methylation of a lysine residue, such as methylation of a histone 3 lysine residue, in particular H3K27Me3, H3KMe2, H3K4Me2 or H3K36Me3. In one embodiment, the histone post translational modification is acetylation of a lysine residue, such as acetylation of a histone 3 lysine residue, in particular H3K9Ac, H3K14Ac or H3K27Ac. In one embodiment, the histone post translational modification is phosphorylation of a serine residue, such a phosphorylation of an isoform X of histone 2A serine residue, in particular pH2AX or phosphorylation of a histone 3 serine residue, such as H3S10Ph. In one embodiment, the histone post translational modification is citrullination of an arginine residue, such as citrullination of a histone 3 arginine residue, in particular H3R8Cit.

In one embodiment, the nucleosomes measured are citrullinated. In a further embodiment, the histone PTM is H3 citrulline (H3cit) or H4 citrulline (H4cit). In a yet further embodiment, the histone PTM is H3cit. In a yet further embodiment, the histone PTM is H3R8cit.

In one embodiment, the histone is mutated, including a mutation in histone 3, such as a mutation in H3 in which lysine 27 is replaced by a methionine (H3K27M).

A group or class of related histone post translational modifications (rather than a single modification) may also be detected. A typical example, without limitation, would involve a 2-site immunoassay employing one antibody or other selective binder directed to bind to nucleosomes and one antibody or other selective binder directed to bind the group of histone modifications in question. Examples of such antibodies directed to bind to a group of histone modifications would include, for illustrative purposes without limitation, anti-pan-acetylation antibodies (e.g. a Pan-acetyl H4 antibody [H4panAc]), anti-citrullination antibodies or anti-ubiquitin antibodies.

In one embodiment, the epigenetic feature of the nucleosome comprises one or more DNA modifications. In addition to the epigenetic signalling mediated by nucleosome histone isoform and PTM composition, nucleosomes also differ in their nucleotide and modified nucleotide composition. Some nucleosomes may comprise more 5-methylcytosine residues (or 5-hydroxymethylcytosine residues or other nucleotides or modified nucleotides) than other nucleosomes. In one embodiment, the DNA modification is selected from 5-methylcytosine or 5-hydroxymethylcytosine.

In one embodiment, the epigenetic feature of the nucleosome comprises one or more protein-nucleosome adducts or complexes. A further type of circulating nucleosome subset is nucleosome protein adducts. It has been known for many years that chromatin comprises a large number of non-histone proteins bound to its constituent DNA and/or histones. These chromatin associated proteins are of a wide variety of types and have a variety of functions including transcription factors, transcription enhancement factors, transcription repression factors, histone modifying enzymes, DNA damage repair proteins and many more. These chromatin fragments including nucleosomes and other non-histone chromatin proteins or DNA and other non-histone chromatin proteins are described in the art.

In one embodiment, the protein adducted to the nucleosome is selected from: a transcription factor, a High Mobility Group Protein or chromatin modifying enzyme. References to “transcription factor” refer to proteins that bind to DNA and regulate gene expression by promoting (i.e. activators) or suppressing (i.e. repressors) transcription. Transcription factors contain one or more DNA-binding domains (DBDs), which attach to specific sequences of DNA adjacent to the genes that they regulate.

All of the circulating nucleosomes and nucleosome moieties, types or subgroups described herein may be useful in the present invention.

It will be understood that more than one epigenetic feature of cell free nucleosomes may be detected in methods and uses of the invention. Multiple biomarkers may be used as a combined biomarker. Therefore, in one embodiment, the use comprises more than one epigenetic feature of cell free nucleosomes as a combined biomarker. The epigenetic features may be the same type (e.g. PTMs, histone isoforms, nucleotides or protein adducts) or different types (e.g. a PTM in combination with a histone isoform). For example, a post-translational histone modification and a histone variant may be detected (i.e. more than one type of epigenetic feature is detected). Alternatively, or additionally, more than one type of post-translational histone modification is detected, or more than one type of histone isoform is detected.

It will be understood that more than one feature of nucleosomes, NETs or ETs may be detected in methods and uses of the invention. Multiple biomarkers may be used as a combined biomarker. Therefore, in one embodiment, the use comprises more than one feature of nucleosomes as a combined biomarker. The features may be the same type (e.g. PTMs or nucleotides) or different types (e.g. a PTM in combination with a nucleotide sequence). Therefore, in one embodiment, the threshold level is determined using a combination of multiple different nucleosomes, and optionally other chromatin fragments, comprising multiple epigenetic features.

In one embodiment, the nucleosomes are measured using an assay such as an immunoassay, immunochemical, mass spectroscopy, chromatographic, chromatin immunoprecipitation or biosensor method.

In one embodiment, the assay employs a single binding agent. In an alternative embodiment, the assay employs more than one binding agent, e.g. two binding agents. In a further embodiment, the assay is a 2-site immunometric assay employing two binding agents.

In one embodiment, the binding agent is directed to a histone, nucleosome core, DNA epitope or a protein adducted to a nucleosome.

In one embodiment, the method of measuring the threshold level of nucleosomes comprises contacting the body fluid sample with a solid phase comprising a binding agent that detects cell free nucleosomes or a component thereof, and detecting binding to said binding agent.

In a further embodiment, the method of measuring the threshold level of nucleosomes comprises: (i) contacting the sample with a first binding agent which binds to an epigenetic feature of a cell free nucleosome or a component thereof (e.g. histone isoform H3.1); (ii) contacting the sample bound by the first binding agent in step (i) with a second binding agent which binds to cell free nucleosomes or a component thereof; and (iii) detecting or quantifying the binding of the second binding agent in the sample.

Detecting or measuring the threshold level of nucleosomes may be performed using one or more reagents, such as a suitable binding agent. In one embodiment, the one or more binding agents comprises a ligand or binder specific for the desired marker on the nucleosome, e.g. H3.1, or a structural/shape mimic of the biomarker or component part thereof. In one embodiment, the binding agent is a chromatin protein. In an alternative embodiment, the binding agent is an antibody. It will be clear to those skilled in the art that the terms “antibody”, “binder” or “ligand” in regard to any aspect of the invention is not limiting but intended to include any binder capable of binding to particular molecules or entities and that any suitable binder can be used in the method of the invention.

Methods of detecting nucleosomes are known in the art. In one embodiment, the ligands or binders of the invention include naturally occurring or chemically synthesised compounds, capable of specific binding to the desired target. A ligand or binder may comprise a peptide, an antibody or a fragment thereof, or a synthetic ligand such as a plastic antibody, or an aptamer or oligonucleotide, capable of specific binding to the desired target. The antibody can be a polyclonal, oligoclonal, monoclonal antibody or a fragment thereof. It will be understood that if an antibody fragment is used then it retains the ability to bind the biomarker so that the biomarker may be detected (in accordance with the present invention). A ligand/binder may be labelled with a detectable marker, such as a luminescent, fluorescent, enzyme or radioactive marker; alternatively or additionally a ligand according to the invention may be labelled with an affinity tag, e.g. a biotin, avidin, streptavidin or His (e.g. hexa-His) tag. Alternatively, ligand binding may be determined using a label-free technology for example that of ForteBio Inc.

Identification and/or quantification of nucleosomes may be performed by detection of the nucleosome or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length. It is noted in particular that peptides of the same or related sequence to that of histone tails are particularly useful fragments of histone proteins.

For example, detecting and/or quantifying can be performed using an immunological method, such as an immunoassay. Immunoassays include any method employing one or more antibodies or other specific binders directed to bind to the biomarkers defined herein. Immunoassays include 2-site immunoassays or immunometric assays employing enzyme detection methods (for example ELISA), fluorescence labelled immunometric assays, time-resolved fluorescence labelled immunometric assays, chemiluminescent immunometric assays, immunoturbidimetric assays, particulate labelled immunometric assays and immunoradiometric assays as well as single-site immunoassays, reagent limited immunoassays, competitive immunoassay methods including labelled antigen and labelled antibody single antibody immunoassay methods with a variety of label types including radioactive, enzyme, fluorescent, time-resolved fluorescent and particulate labels.

In another example, detecting and/or quantifying can be performed by one or more method(s) selected from the group consisting of SELDI (-TOF), MALDI (-TOF), a 1-D gel-based analysis, a 2-D gel-based analysis, Mass spectrometry (MS), reverse phase (RP) LC, size permeation (gel filtration), ion exchange, affinity, HPLC, UPLC and other LC or LC MS-based techniques. Appropriate LC MS techniques include ICAT@(Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods involving identification and/or quantification of nucleosomes can be performed on bench-top instruments, or can be incorporated onto disposable, diagnostic or monitoring platforms that can be used in a non-laboratory environment, e.g. in the physician's office or at the subject's bedside.

### Sample

The sample may be any body fluid sample taken from a subject including, without limitation, cerebrospinal fluid (CSF), whole blood, blood serum, plasma, menstrual blood, endometrial fluid, urine, saliva, or other bodily fluid (stool, tear fluid, synovial fluid, sputum), breath, e.g. as condensed breath, or an extract or purification therefrom, or dilution thereof. In a preferred embodiment, the body fluid sample is selected from blood, serum or plasma.

Biological samples also include specimens from a live subject, or taken post-mortem. The samples can be prepared, for example where appropriate diluted or concentrated, and stored in the usual manner. For example, the samples may be used fresh, or frozen and stored until analysis is required. It will be understood that methods and uses of the present invention find particular use in blood, serum or plasma samples obtained from a patient. In one embodiment, the sample is a blood or plasma sample. In a further embodiment, the sample is a serum sample. In a further embodiment both serum and plasma samples are used for the measurement of different members of an assay panel.

References to “subject” or “patient” are used interchangeably herein. The subject may be a human or an animal subject. In one embodiment, the subject is a human. In one embodiment, the subject is a (non-human) animal. In one embodiment, the subject is a non-human mammal, such as a dog, mouse, rat or horse, in particular a dog. In a further embodiment, the animal is a companion animal (also referred to as a pet or domestic animal). Companion animals include, for example dogs, cats, rabbits, ferrets, horses, cows, or the like. In particular, the companion animal is a dog or cat, particularly a dog. The use, panels and methods described herein may be performed in vitro, in vivo or ex vivo.

### Threshold Level

The present invention in certain embodiments involves measuring nucleosomes for a threshold level and based on this determining whether the associated DNA meets a required level which is suitable for DNA analysis. The present invention in certain embodiments involves measuring nucleosomes and based on the measured level determining the determining the volume of body fluid sample required to obtain an amount of DNA for DNA sequence analysis using the level of nucleosomes measured and optionally obtaining a further body fluid sample of at least the volume previously determined.

It is typically recommended that at least 1 ng, and preferably up to 30 ng, of DNA is used to obtain reliable sequencing results. The inventors have found that using 1-2 ml of a 10-20 ng/ml DNA solution typically provides results with >90% alignment of human sample results with human sequence databases with high reproducibility.

Typically, library preparations comprise ≥10 ng of DNA. Alternatively, lower starting amounts or concentrations of DNA may be used (for example 10 pg of DNA in the form of 0.5-1 ml of a 0.2 ng/ml solution). However, this may lead to read duplication (i.e. obtaining multiple results for the same fewer sequences present due to amplification).

As indicated previously, the vast majority of cfDNA present in a body fluid sample occurs as nucleoprotein complexes in the form of mononucleosomes, oligonucleosomes or polynucleosomes (Sanchez et al, 2021). The present invention employs the inventive concept that measurement of nucleosomes in a sample will give a result that approximates to the level of DNA present.

The molecular weight of a nucleosome is approximately 200 kD. The molecular weight of 147 bp DNA is approximately 96 kD but will vary with DNA sequence. Therefore, as an approximation, a nucleosome comprises around 50% DNA by mass.

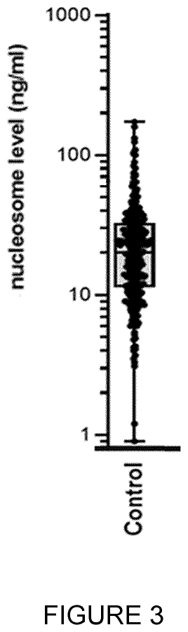

H3.1-nucleosome levels we have measured for frozen samples obtained from healthy subjects are typically <65 ng/ml (FIG. 3) and we have observed that elevated levels are likely to be pathological. However, the relevant ctDNA component of plasma cfDNA may be 1% or less in a cancer sample and the ctDNA of interest in a sample containing 65 ng/ml nucleosomes may therefore be 0.325 ng/ml or less.

Therefore, in one embodiment a threshold nucleosome level is used as a minimal level at which DNA sequencing of a sample is indicated or is used to determine the volume of sample required to obtain sufficient DNA material for sequencing. For example, approximately 3 ml of a sample containing 65 ng/ml may yield approximately 1 ng ctDNA.

Using a 1% ctDNA level as a metric, a sample containing 100 ng/ml nucleosomes (by DNA composition) may contain some 50 ng/ml of cfDNA comprising of the order of 0.5 ng/ml ctDNA. As described above, it is typically recommended that at least 1 ng, and preferably up to 30 ng, of DNA is used to obtain reliable sequencing results.

Therefore, in one embodiment a threshold level is used as a minimal level likely to yield sufficient DNA material for sequencing. For example, approximately 2 ml of a sample containing 100 ng/ml nucleosomes may yield about 100 ng cfDNA which may comprise about 1 ng ctDNA.

The threshold level and/or the actual nucleosome result may be used further to estimate a minimal or optimal sample volume required to for sequencing. For example a minimum volume of 1 ml of a sample containing 200 ng/ml nucleosomes or a minimum of volume of 4 ml of a sample containing 50 ng/ml nucleosomes may be used to obtain a minimum of 100 ng cfDNA comprising of the order of 1 ng ctDNA for sequencing. In many cases the optimal sample volume to be used for sequencing may be higher than the minimum, for example to obtain 5 ng or 10 ng or more of relevant DNA, for example ctDNA, for sequencing to increase the reliability and accuracy of sequencing.

Thus, in an embodiment of the invention, the threshold level of nucleosomes is 10 ng/ml to 3000 ng/ml, 20 to 3000 ng/ml, 100 ng/ml to 3000 ng/ml or 1000 ng/ml to 3000 ng/ml.

In another embodiment of the invention, the threshold level of nucleosomes is 10 ng/ml to 2000 ng/ml of DNA, 20 ng/ml to 2000 ng/ml, 100 ng/ml to 2000 ng/ml of DNA or 1000 ng/ml to 2000 ng/ml of DNA.

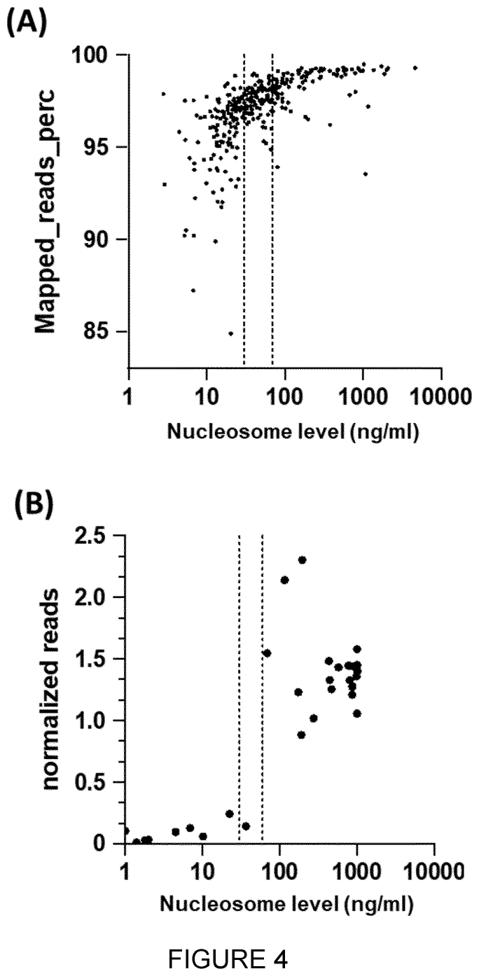

In another embodiment of the invention, the threshold level of nucleosomes is at least 30 ng/ml, such as at least 40 ng/ml, at least 50 ng/ml, at least 60 ng/ml, at least 70 ng/ml, at least 80 ng/ml, at least 90 ng/ml or at least 100 ng/ml. The results shown in FIG. 4 indicate that a nucleosome threshold level of about 50 ng/ml was able to identify sample suitable for sequencing.

In one embodiment of the invention the threshold level of nucleosomes is at least 10 ng/ml. In a particular embodiment of the invention the threshold level of nucleosomes is at least 100 ng/ml. In another particular embodiment of the invention the threshold level of nucleosomes is at least 1000 ng/ml.

In another embodiment of the invention, the required level of DNA for DNA sequencing is 0.1 ng to 30 ng of DNA, 0.2 ng to 30 ng DNA, 1 ng to 30 ng of DNA or 10 ng to 30 ng of DNA.

In another embodiment of the invention, the required level of DNA for DNA sequencing is 0.1 ng to 20 ng of DNA, 0.2 ng to 20 ng DNA, 1 ng to 20 ng of DNA or 10 ng to 20 ng of DNA.

In one embodiment of the invention, the required level of DNA for DNA sequencing is at least 0.1 ng of DNA. In another embodiment of the invention, the required level of DNA for DNA sequencing is at least 1 ng of DNA. In another embodiment of the invention, the required level of DNA for DNA sequencing is at least 10 ng of DNA. In a particular embodiment of the invention the required level of DNA when the sequencing does not involve PCR is at least 1 ng of DNA.

### Extraction

Extraction of cfDNA from body fluid samples, such as blood, serum or plasma, for analysis of ctDNA is usually performed using commercially available DNA extraction products; however, any convenient method may be used. Such extraction methods claim high recoveries of circulating DNA (>50%) and some products (for example, the QIAamp Circulating Nucleic Acid Kit produced by Qiagen) are claimed to extract DNA fragments of small size. Typical sample volumes used are in the range 1-5 mL of plasma.

### Choice of DNA Sequencing Method

The blood of cancer patients contains circulating tumour DNA (ctDNA) which is thought to originate from the release of chromatin fragments or nucleosomes into the circulation from dying or dead cancer cells. Tumour derived cfDNA or ctDNA circulates as small DNA fragments consistent with the size expected for mononucleosomes. Investigation of matched blood and tissue samples from cancer patients shows that cancer associated mutations, present in a patient's tumour (but not in his/her healthy cells) can also be present in ctDNA in blood samples taken from the same patient (Newman et al, 2014). Similarly, DNA sequences that are differentially methylated (epigenetically altered by methylation of cytosine residues) in cells can also be detected as methylated sequences in cfDNA in the circulation. In addition, the proportion of cell-free circulating DNA (cfDNA) that is comprised of ctDNA is related to tumour burden so disease progression may be monitored both quantitatively by the proportion of ctDNA present and qualitatively by its genetic and/or epigenetic composition. Analysis of ctDNA can produce highly useful and clinically accurate data pertaining to DNA originating from all or many different clones within the tumour and which integrates the tumour clones spatially. Moreover, repeated sampling over time is a much more practical and economic option. Analysis of ctDNA has the potential to revolutionize the detection and monitoring of tumours, as well as the detection of relapse and acquired drug resistance at an early stage for selection of treatments for tumours through the investigation of tumour DNA without invasive tissue biopsy procedures and also the ability to monitor which types of cells are dying following treatment. Such ctDNA tests may be used to investigate all types of cancer associated DNA abnormalities (e.g. point mutations, nucleotide modification status, translocations, gene copy number, micro-satellite abnormalities, aneuploidy and DNA strand integrity) and would have applicability for routine cancer screening, regular and more frequent monitoring and regular checking of optimal treatment regimens (Zhou et al, 2012).

Gene copy number refers to the number of copies of a particular gene present in the genome of an individual. Genomic instability is characteristic of cancer and copy number alterations are a prevalent mutation in cancer. Copy number alterations include insertions, deletions, and duplications of segments of DNA leading to changes to chromosome structure that result in a gain or loss in copies of sections of DNA. Amplifications and deletions can occur to part of a gene, entire genes, large sections of chromosomes, including an entire arm as well as the whole chromosomes itself.

It will be appreciated that any method of DNA sequencing may be used with methods of the invention. In preferred embodiments the sequencing method is a sequencing by synthesis (SBS) method or a nanopore sequencing method.

SBS and nanopore sequencing are two commonly used sequencing methods. SBS is the method of choice for the detection of focal alterations including, for example, point mutations, codon/exon insertions and deletions where high coverage is required.

Nanopore sequencing is more appropriate for longer range alterations such as aneuploidy and chromosomal arm amplifications or deletions particularly where rapid results are required. One advantage we have found for nanopore sequencing is to provide direct methylated and/or hydroxymethylated DNA sequence results for cfDNA in real time (with no chemical pretreatment). Nanopore sequencer instruments can also be small, low cost and suitable for use near to the patient. Moreover, when sufficient data has been obtained in real time, sequencing can be terminated avoiding the unnecessary use of further reagents leading to economy of use. Furthermore, nanopore sequencers are small and so can be used broadly and closer to the patient.

Many DNA analysis methods are known in the art including, without limitation, Sanger sequencing, next generation sequencing (NGS) (targeted or whole genome), circular consensus sequencing (CCS), sequencing by binding (SBB), PCR, BEAMing, digital PCR, isothermal DNA amplification, cold PCR (co-amplification at lower denaturation temperature-PCR), MAP (MIDI-Activated Pyrophosphorolysis), pyrosequencing, methylation sensitive restriction enzyme (MSRE) digestion and PARE (personalized analysis of rearranged ends). In one embodiment, said sequencing comprises Next Generation Sequencing (NGS). In one embodiment, said sequencing comprises sequencing the whole genome. In an alternative embodiment, said sequencing comprises sequencing targeted regions of the genome.

SBS is a commonly used current DNA sequencing method and the term NGS normally relates to sequencing by synthesis (for example the sequencing methods employed by Illumina NGS instruments are sequencing by synthesis). The details of SBS sequencing methods employed vary greatly. A typical method involves the fragmentation of DNA strands to be sequenced into short fragments of a few hundred base pairs. A library of DNA fragments is prepared for sequencing by ligating or tagmenting sequencing adapters to the fragments. Typically, the library is amplified using PCR methods (PCR free methods can also be used). Sequencing of the fragments is performed by synthesizing a new complementary strand to strands in the library typically using 4 fluorescent nucleotides (adenine, thymine, cytosine and guanine) each labelled with a different detectable fluorophore. There is also a rapid-run version that only uses 2 colours but still measures all 4 bases (the colours individually, both and neither). Typically the ends of the fragments only are sequenced. The desired DNA sequence is then generated from the results using bioinformatics. The sequence data is available at the end of the process. SBS is the most commonly used sequencing modality because it can be used to generate high DNA coverage results (e.g. 30× coverage or greater) and produces highly accurate sequence data. However, SBS cannot provide sequence data relating to non-standard nucleotides such as 5-methylcytosine without further chemical processing such as bisulfite conversion and typically requires 2 or 3 days with results available only after completion of sequencing and bioinformatic analysis. The cost of SBS depends on the depth of sequencing required and the number of samples sequenced, with lower costs when large numbers of samples are sequenced in parallel. Recent cost estimates for DNA sequencing for clinical purposes range up to £6841 per cancer case for matched tumor and germline samples) and up to £7050 per rare disease case comprising three samples. (Schwarze et al. (2020)). Clinical sequencing may be applied to patient plasma or other body fluid samples that contain insufficient DNA to generate reliable, accurate, robust and reproducible sequence data. Sequencing of plasma samples that contain inadequate DNA levels may produce poor quality and/or misleading results and is wasteful and expensive.

In one embodiment, said sequencing comprises a nanopore sequencing method. Nanopore DNA sequencing (for example the sequencing methods employed by Oxford Nanopore Technology DNA instruments) is becoming more commonly employed by workers in the field. In short nanopore sequencing involves the passage of DNA strands through electrically charged nanopores. When a nucleotide passes through a nanopore an electrical disturbance characteristic of the nucleotide is induced and this is detected by a sensor connected to the nanopore. Nanopore sequencing typically produces lower coverage results and less accurate DNA sequence results than SBS but has a number of advantages including the ability to sequence long DNA chains without fragmentation, the ability to directly sequence non-standard nucleotides in addition to adenine, thymine, cytosine and guanine (for example 5-methylcytosine and 5-hydroxymethylcytosine) and the generation of sequence data in real time and without library amplification. This facilitates the obtaining of sequence data in a shorter time of a few hours or less. Nanopore sequencing is expensive if used for high coverage sequencing of whole genomes but more economic for shallow sequencing (e.g. 1× coverage) of cfDNA fragments.

In one embodiment, said sequencing method is an epigenetic sequencing method for a methylated or hydroxymethylated DNA sequence

Analysis of cfDNA

Fragmentomics is based on the premise that the sequences of cfDNA fragments found in plasma are protected from digestion by binding to protein which may be histone in nature, as in nucleosomes, or may be by other proteins such as transcription factors. Other DNA sequences not protein protected are largely digested and hence absent from the plasma. The fragmentation patterns obtained by sequencing an individual's cfDNA can be built into a map of nucleosome and other protein occupancy. DNA occupancy patterns are cell or tissue specific and cfDNA occupancy maps obtained by sequencing of plasma samples can be compared to cellular DNA occupancy patterns determined for cell types by nuclease-accessible site analysis (also known as DNase hypersensitivity analysis) or ATAC-Seq methods (assay for transposase-accessible chromatin with sequencing). The comparison can be used to determine the origin of cfDNA. Using this method, the cfDNA of healthy subjects has been found to correlate strongly with the occupancy patterns of lymphoid and myeloid cell lines. Sequencing of cfDNA from late-stage cancer patients showed additional occupancy patterns that correlated most strongly with occupancy maps of cancer cell lines, often matching the anatomical origin of the patient's cancer (see for example US2022028494 and Snyder et al, 2016). One example application of fragmentomics methods is to establish the tissue or cell of origin of cfDNA released concomitant to a disease condition, for example to establish the tissue affected by a cancer.

Genomic DNA-methylation patterns are similarly cell type specific. DNA-methylation analysis involves sequencing of DNA where, in addition to the information comprising the sequence of the 4 standard nucleotides (adenine, thymine, cytosine and guanine or A, T, C and G), sequence information relating to the DNA positions at which cytosine is methylated (5-methylcytosine or 5mc) is also determined. Typically, bisulfite conversion sequencing methods are used in which cfDNA is extracted from plasma and then treated with bisulfite to convert unmodified cytosine residues to uracil. Sequencing, can then be applied to determine the methylated gene sequence. The tissue or cell of origin of cfDNA may be established by DNA-methylation sequencing and comparison of observed methylation patterns with those established as characteristic of cell types. One example application of cfDNA-methylation sequencing is to establish the tissue or cell of origin of cfDNA released concomitant to a disease condition, for example to establish the tissue affected by a cancer.

In one embodiment, the methods of the invention comprise using the sequencing to identify the tissue of origin or the cell of origin of the cfDNA in the sample.

Therefore, according to one aspect of the present invention there is provided a method for the analysis of DNA to identify the tissue of origin or the cell of origin of the cell free DNA (cfDNA) in the sample in a body fluid sample comprising:
(a) measuring the level of nucleosomes in a body fluid sample;
(b) determining if the level of nucleosomes measured in step (a) meets a threshold level of nucleosomes;
(c) optionally extracting the DNA from the sample if the level of nucleosomes measured in step (b) meets the threshold level of nucleosomes;
(d) sequencing the DNA present in the sample if the level of nucleosomes measured in step (b) meets the threshold level and
(e) analysing the sequenced DNA to identify the tissue of origin or the cell of origin of the cfDNA in the sample.

Plasma and other body fluid samples obtained from healthy subjects have low levels of cell free nucleosomes. Higher levels are reported for a number of cancer and inflammatory conditions. Some patients with solid cancers (for example lung cancer, breast cancer, prostate cancer, colorectal cancer or any solid cancer) have high levels of circulating cell free nucleosomes (Holdenrieder et al, 2001). We have observed particularly high levels in subjects (several hundreds of ng/ml) diagnosed with myeloproliferative diseases such as leukaemia, lymphoma and myeloma as well as proliferative diseases of the vasculature such as canine lymphoma and hemangiosarcoma. We have also observed very high levels (up to several thousands of ng/ml) in conditions involving inappropriately high cytokine production and highly elevated levels of ETs and/or NETs such as sepsis and severe cases of COVID-19 infection (Morimont et al; 2022)

Therefore, when a high level of cell free nucleosomes is observed in a sample obtained from a subject, there is a problem related to the interpretation of the result because the cell free nucleosomes may be of cancer or NETosis, NETs or ETs origin. A high NETs or ETs level would be indicative of a NETosis associated disorder such as sepsis. A high level of cell free nucleosomes of another tissue origin may be indicative of a cancer of that tissue. For example a high level of nucleosomes of lung tissue origin may be indicative of lung cancer.

It may be possible to distinguish sepsis or other NETosis related conditions from myeloproliferative or other cancer disease in some subjects on clinical grounds. Similarly, it may be possible to distinguish between different cancer types and identify the tissue affected by cancer (for example a lung cancer, colorectal cancer, lymphoma, leukaemia etc) in some subjects on clinical grounds. However, it would be advantageous to have a confirmation of the cellular source of the elevated nucleosome level as comprising mostly, or a significant proportion of, nucleosomes of neutrophil origin, confirming a NETotic origin, or other tissue origin, for example a lung cell origin indicating a likely lung cancer disease case.

One method for the identification of the tissue of origin of cell free nucleosomes in a body fluid sample is methylated DNA sequencing. Nanopore sequencing is particularly suited to the sequencing of DNA strands containing 5-methylcytosine residues and the sequence of a strand in terms of its adenine, thymine, guanine, cytosine and 5-methylcytosine may be determined directly without any chemical pre-treatment of the 5-methylcytosine residues and optionally without any amplification. The DNA methylation pattern of chromatin is characteristic of cell type and the cfDNA methylation pattern obtained for a sample can be compared to known tissue patterns to identify the cfDNA tissue(s) of origin (Barefoot et al, 2021).

We measured the level of nucleosomes containing histone H3.1 (H3.1-nucleosomes) present in a sample obtained from a subject diagnosed with sepsis and from a subject diagnosed with a B-cell lymphoma. Both of these samples had highly elevated H3.1-nucleosome levels, so discrimination between the two on the basis of H3.1-nucleosome level alone was not possible. As the H3.1-nucleosome level was elevated, we determined that the sample contained sufficient cfDNA for analysis by sequencing. We extracted DNA from the samples and performed DNA sequencing using a nanopore sequencing method. The proportions of the cfDNA originating from different cell types was derived by comparison of the methylated sequence results for the two samples to known DNA methylation patterns for a variety of different cell types.

The methods of the invention can identify patients with an abnormally high level of nucleosomes, and therefore also of cfDNA, and who therefore are likely to be suffering from a disease condition. The condition will be unknown at this point but samples with elevated nucleosome levels contain sufficient cfDNA for sequencing purposes. Moreover, the amount or volume of sample required for sequencing can be ascertained. The cfDNA present in the sample may be extracted and sequenced by any DNA sequencing method. The sequence data produced may be used to identify the tissue or tissues of origin of the cfDNA and this information may be used to diagnose a medical condition of the subject, select suitable therapies for treatment of the subject or monitor the condition of the subject through repeated sample analysis.

Therefore, in one embodiment of the invention, there is provided a method for the determination of the tissue(s) of origin of cfDNA in a blood, serum or plasma sample obtained from a subject identified as having an elevated level of nucleosomes (and hence also an elevated level of cfDNA), which comprises the steps of:
(a) measuring the level of nucleosomes in the sample;
(b) using the level of nucleosomes measured in step (a) to determine if the sample contains an elevated level of nucleosomes;
(c) optionally extracting cfDNA from the sample;
(d) sequencing the cfDNA present in the sample; and
(e) using the sequence data to identify the tissue(s) of origin of the cfDNA in the sample.

As described hereinbefore, the method may include determining the volume of sample required to obtain sufficient DNA for reliable DNA sequence analysis. Therefore, the method may additionally comprise: (i) using the level of nucleosomes measured in step (a) to determine the volume of sample required to obtain sufficient DNA for DNA sequence analysis; and optionally obtaining the volume of sample determined in step (i) (i.e. if further sample is required).

The method will now be illustrated by the following examples.

### EXAMPLES

### Example 1

EDTA plasma samples were obtained from 2 subjects diagnosed with cancer. One subject was diagnosed with prostate cancer and one with B-cell non-Hodgkin's lymphoma (NHL). The plasma samples were analysed for intact cell free nucleosomes containing histone isoform H3.1 using a manual ELISA method available from Belgian Volition SRL as per the manufacturer's instructions. Briefly, calibrant or sample was diluted in assay buffer and incubated in a microtiter well coated with an anti-histone H3.1 antibody for 150 minutes with shaking. The diluted sample or calibrant was discarded and the wells were washed 3 times with a wash buffer. A solution of horse radish peroxidase labelled anti-nucleosome antibody was added and the wells were incubated a further 90 minutes with shaking. The labelled antibody was discarded and the wells were washed 3 times with a wash buffer. A coloured enzyme substrate solution (3,3′,5,5′-Tetramethylbenzidine) was added and the wells were incubated a further 20 minutes with shaking. A STOP solution (1M HCl) was added to stop the enzyme reaction and the light absorption at 450 nm was measured. The concentration of intact cell free nucleosomes containing histone isoform H3.1 in the unknown samples was interpolated from a standard curve.

The prostate cancer sample was found to have a H3.1-nucleosome level of 39.3 ng/ml. This level is within the range of levels expected for healthy subjects as shown in FIG. 3. The NHL sample was found to have a H3.1-nucleosome level of 743.5 ng/ml. This level is elevated compared to the range of levels expected for healthy subjects.

DNA was extracted from 1 ml of the plasma samples and libraries were prepared using Oxford Nanopore Technology's Ligation Sequencing kit. The DNA was sequenced using a nanopore sequencing method and the results analysed using iChor CNA for copy number alterations (see https://github.com/broadinstitute/ichorCNA and Adalsteinsson et al, 2017).

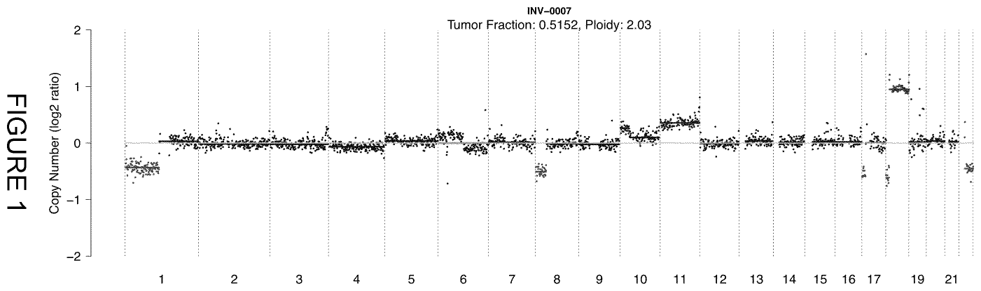

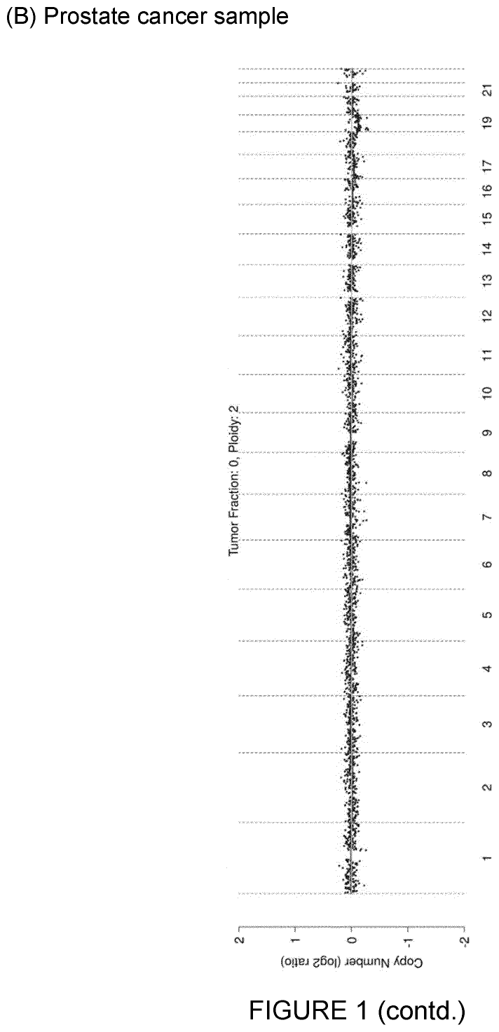

The NHL sample with a high measured nucleosome level was found to contain amplification of chromosomes 11 and 18 and partial amplification of chromosome 10. Similarly, deletions were found to parts of chromosomes 1, 8, 16, 17 and 22 (FIG. 1). These observations are consistent with reports that copy number alterations are characteristic of NHL diseases (Conde et al; 2014).

In contrast, no copy number alterations (CNA) were observed for the prostate cancer sample with a low level of nucleosomes. As prostate cancer is associated with a well-known repertoire of copy number alterations (Grist et al, 2022), we conclude that the low level of cfDNA present in the prostate cancer sample, as indicated by the low level of cell free nucleosomes present, was insufficient to enable CNA detection. We also conclude, (i) that the NHL sample was indicated as suitable for DNA sequencing by the high nucleosome level measured and (ii) that sequencing of the prostate cancer sample was contra-indicated by the low nucleosome level measured. We further conclude that methods of the invention may be used to identify samples suitable for DNA sequencing, and to detect the presence of a cancer in a subject with an elevated plasma nucleosome level.

### Example 2

The sequence data obtained for the prostate and NHL samples described in EXAMPLE 1 above, was analysed for methylated DNA sequences. The methylated DNA sequence data was compared to databases of known methylated DNA sequences for a variety of cell types to determine the cell types of origin of the cfDNA in the samples using methods known in the art (see for example, Moss et al; 2018). The results obtained for the NHL sample indicated that the cfDNA present comprised 51.5% of DNA of B-cell origin. This is consistent with the diagnosis of B-cell lymphoma and indicates that around 50% of the cfDNA present was of tumour origin. In contrast, no cfDNA of prostate origin was detected in the prostate cancer sample indicating that insufficient prostate derived DNA to enable such detection was present in the sample. We conclude, that the NHL sample was correctly identified as suitable for sequencing by the high nucleosome level measured and that the sequencing of the prostate cancer sample was correctly contra-indicated by the low nucleosome level measured.

### Example 3

We have previously shown that sepsis is associated with high plasma cell free nucleosome levels (WO2021186037). In order to further confirm the findings of EXAMPLES 1 and 2 above using a sample with a high nucleosome level from a non-cancer disease, a further EDTA plasma sample was obtained from a subject diagnosed with sepsis. This sample was analysed for nucleosome level and copy number alterations as described in EXAMPLE 1.

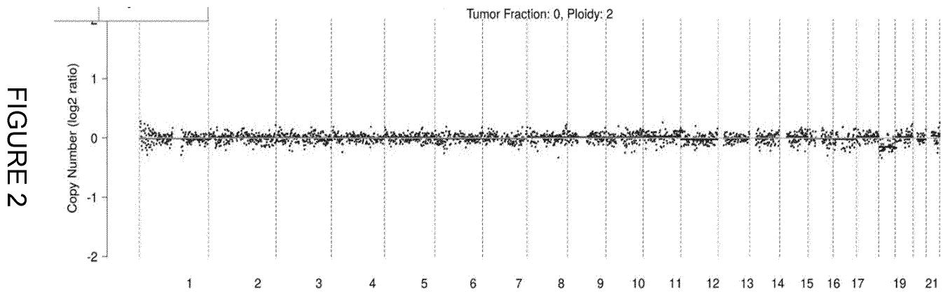

The sepsis sample was found to contain a highly elevated level of 3281.3 ng/ml of H3.1-nucleosomes. As shown by the successful CNA analysis of the NHL sample, this nucleosome level is higher than that required for CNA analysis and hence indicated that the sample was suitable for DNA sequencing. However, no copy number alterations were observed for this sample (FIG. 2). We conclude that DNA sequencing of the sepsis sample was indicated by the H3.1-nucleosome level and that the finding of an absence of copy number alterations in the sample is consistent with absence of a cancer disease in the subject.

### Example 4

The sequence data obtained for the sepsis sampled described in EXAMPLE 3 above, was analysed for methylated DNA sequences as described in EXAMPLE 2. The results obtained for the sepsis sample indicated that the cfDNA present comprised approximately 70% of DNA of neutrophil origin. This is consistent with reports that the nucleosomes present in subjects with sepsis are derived predominantly from neutrophil extracellular trap material (Morimont et al; 2022).

### Example 5

Plasma samples were obtained from humans and canines and cell free DNA was sequenced either using paired-end next generation sequencing (for human samples) or nanopore genome sequencing (for canine samples). For each human sample, the percentage of reads mapping was calculated. For each canine sample, the total number of sequencing reads generated was calculated, and divided by the average number of sequencing reads for all samples on the same flowcell (reads/FCmean) to provide a normalized read count. The read value was compared to the H3.1-nucleosome level for the same sample (log scale) measured using the method described in EXAMPLE 1, above. The results are shown in FIG. 4. Nucleosome threshold levels were able to identify samples most suited for DNA sequencing.

We conclude that the method of the invention may be used to identify samples with an elevated level of cell free nucleosomes as suitable for DNA sequencing. We also conclude that the result of such DNA sequencing may be used to identify whether an elevated level of nucleosomes present in a sample has a cancer or a non-cancer origin. We further conclude that methods of the invention may be used to identify the cell type or tissue of origin of cfDNA in a subject with an elevated level of nucleosomes.

### REFERENCES

Adalsteinsson et al (2017) Nature Comms. 8: 1324. doi: 10.1038/s41467-017-00965-y
Ausio (2001) Biochem. Cell Bio. 79: 693
Barefoot et al (2021) Front. Genet. doi: 10.3389/fgene.2021.671057
Church et al. (2014) Gut 63:317-325. doi:10.1136/gutjnl-2012-304149
Conde et al. (2014) PLoS One 9(8): e105382. doi: 10.1371/journal.pone.0105382.
Crowley et al. (2013) Nat Rev. Clin. Oncol. 10: 472-484
Grist et al. (2022) Genome Med. 14:102. doi: 10.1186/s13073-022-01080-4
Holdenrieder & Stieber (2009) Crit Rev. Clin. Lab. Sci. 46(1): 1-24
Holdenrieder et al. (2001) Int. J. Cancer 95: 114-120
Jung et al. (2010) Clin. Chim. Acta 411(21-22): 1611-24
Mariño-Ramirez et al. (2011) Database (Oxford) 2011: bar048.
Morimont et al. (2022) Biomolecules 12: 1038. doi: 10.3390/biom12081038.
Moss et al. (2018) Nature Communications 9: 5068. doi: 10.1038/s41467-018-07466-6
Newman et al. (2014) Nat Med. 20(5): 548-54
Salgame et al. Nucleic Acids Res. (1997) 25(3): 680-681
Sanchez et al (2021) JCI Insight 8; 6(7):e144561. doi: 10.1172/jci.insight144561.
Schwarze et al. (2020) Genetics Med. 22: 85-94, doi: 10.1038/s41436-019-0618-7
Snyder et al. (2016) Cell 164(1-2): 57-68
van Nieuwenhuijze et al. (2003) Ann. Rheum. Dis. 62: 10-14
Zhao and Garcia (2015) Cold Spring Harb. Perspect. Biol., 7: a025064
Zhou et al. (2012) Seminars in Oncology. 39: 440-448, doi 10.1053/j.seminoncol.2012.05.013

1. A method for the analysis of DNA in a body fluid sample, comprising:
(a) measuring a level of nucleosomes in a body fluid sample;
(b) determining if the level of nucleosomes measured in step (a) meets a threshold level of nucleosomes;
(c) optionally extracting DNA from the sample if the level of nucleosomes measured in step (b) meets the threshold level of nucleosomes; and
(d) sequencing DNA present in the sample if the level of nucleosomes measured in step (b) meets the threshold level.

2. The method of claim 1, wherein the DNA is sequenced to identify a tissue of origin or a cell of origin of the DNA in the sample.

3. The method of claim 1, further comprising determining a volume of body fluid sample required to obtain an amount of DNA for DNA sequence analysis using the level of nucleosomes measured in step (a); and optionally obtaining a further body fluid sample of at least the volume determined in step (c).

4. The method of claim 1, wherein the threshold level of nucleosomes is between about 10 ng/ml to 3000 ng/ml.

5. The method of claim 1, wherein the threshold level of nucleosomes is at least about 50 ng/ml.

6. A method for the analysis of DNA in a body fluid sample, comprising:
(a) measuring a level of nucleosomes in sample obtained from a body fluid;
(b) determining a volume of body fluid sample required to obtain a required level of DNA for DNA sequencing using the level of nucleosomes measured in step (a);
(c) obtaining a body fluid sample of at least the volume determined in step (b);
(d) optionally extracting DNA from the sample; and
(e) sequencing the DNA present in the sample.

7. The method of claim 6, wherein the required level of DNA for DNA sequencing is between about 0.1 ng to 30 ng of DNA.

8. (canceled)

9. The method of claim 6, wherein the required level of DNA when the sequencing does not involve PCR is at least about 1 ng of DNA.

10. The method of claim 6, further comprising using the sequencing to identify a tissue of origin or a cell of origin of cell free DNA in the sample.

11. The method of claim 6, further comprising obtaining the body fluid sample from a subject.

12. The method of claim 1, wherein the body fluid sample is a blood, serum or plasma sample.

13. The method of claim 1, wherein the DNA is cell free DNA (cfDNA).

14. The method of claim 1, wherein the nucleosomes measured are H3.1-containing nucleosomes or H3.3-containing nucleosomes or wherein the nucleosomes measured are citrullinated.

15. (canceled)

16. The method of claim 1, wherein the nucleosomes are measured using an assay selected from the group consisting of an immunoassay, an immunochemical assay, mass spectroscopy, chromatography, chromatin immunoprecipitation and a biosensor method.

17. The method of claim 16, wherein the assay employs a single binding agent or wherein the assay is a 2-site immunometric assay employing two binding agents.

18. (canceled)

19. The method of claim 17, wherein the binding agent binds a histone, a nucleosome core, a DNA epitope or a protein adducted to a nucleosome.

20. The method of claim 17, wherein the binding agent is a chromatin protein or an antibody.

21. (canceled)

22. The method of claim 1, wherein said sequencing comprises a nanopore sequencing method or Next Generation Sequencing (NGS).

23. (canceled)

24. The method of claim 1, wherein said sequencing method is an epigenetic sequencing method for a methylated or hydroxymethylated DNA sequence.

25. The method of claim 1, wherein the sample is obtained from a human or an animal subject.
